SEQUENCE LISTING (14 sequences)

SEQ ID NO: 1 - DNA, 19 bp, Artificial Sequence
  synthetic oligonucleotide
ggaaacagctatgaccatg

SEQ ID NO: 2 - DNA, 19 bp, Artificial Sequence
  synthetic oligonucleotide
gtaatacgactcactatag

SEQ ID NO: 3 - DNA, 19 bp, Artificial Sequence
  synthetic oligonucleotide
gtgccagcagccgcggtaa

SEQ ID NO: 4 - DNA, 16 bp, Artificial Sequence
  synthetic oligonucleotide
gggcatcacagacctg

SEQ ID NO: 5 - DNA, 20 bp, Artificial Sequence
  synthetic oligonucleotide
ggcgctcacgcgtcactgct

SEQ ID NO: 6 - DNA, 18 bp, Artificial Sequence
  synthetic oligonucleotide
ggctgggacgccgatgat

SEQ ID NO: 7 - DNA, 20 bp, Artificial Sequence
  synthetic oligonucleotide
ggcgctcacgcgtcactgct

SEQ ID NO: 8 - DNA, 18 bp, Artificial Sequence
  synthetic oligonucleotide
ggctgggacgccgatgat

SEQ ID NO: 9 - DNA, 18 bp, Artificial Sequence
  synthetic oligonucleotide
ctggcgggccgttcttcg

SEQ ID NO: 10 - DNA, 22 bp, Artificial Sequence
  synthetic oligonucleotide
ccaacggtcacaggatttcacc

SEQ ID NO: 11 - DNA, 18 bp, Artificial Sequence
  synthetic oligonucleotide
ctggcgggccgttcttcg

SEQ ID NO: 12 - DNA, 22 bp, Artificial Sequence
  synthetic oligonucleotide
ccaacggtcacaggatttcacc

SEQ ID NO: 13 - DNA, 21 bp, Artificial Sequence
  synthetic oligonucleotide
ggatccgatggcgggccgttc

SEQ ID NO: 14 - DNA, 22 bp, Artificial Sequence
  synthetic oligonucleotide
ttcgtcgtgcaacggtcacagg

(19) United States
(12) Patent Application Publication
(10) Pub. No.: US 2007/0031869 A1
(43) Pub. Date: Feb. 8, 2007

(54) Template specific inhibition of PCR

(76) Inventors: Adam M. McCoy, Milpitas, CA (US); Stephen R. Palumbi, Pacific Grove, CA (US)

Correspondence Address: BOZICEVIC, FIELD & FRANCIS LLP, 1900 UNIVERSITY AVENUE, SUITE 200, EAST PALO ALTO, CA, 94303, US

(21) Appl. No.: 11/486,329

(22) Filed: Jul. 12, 2006

Related U.S. Application Data
(60) Provisional application No. 60/698,668, filed on Jul. 12, 2005.

(51) Int. Cl. C12Q 1/68; C12P 19/34

(52) U.S. Cl. 435/6; 435/91.2

(57) ABSTRACT

Template specific inhibition during PCR (TSI-PCR) allows specific inhibition of particular templates without disrupting the amplification of other templates that share amplification primer binding sites. TSI-PCR is achieved by ligation of stop oligos comprising a 3′ modification that prevents extension by DNA polymerases. Stop oligos hybridize to a region of the targeted amplicon downstream of the amplification primer. When the stop oligos are perfectly complementary to the target template, they are ligated onto the extending strand during the extension phase of the amplification cycle. This effectively truncates the extending strand at the site where the stop oligo binds and blocks further extension. The truncated products are not full-length and cannot be extended, and therefore do not serve as additional templates during sunsequent rounds of amplification. This results in substantial inhibition over multiple amplification cycles, but only for templates that match the stop oligos.

In a variety of different fields of biological research, methods for quantitating nucleic acid sequences have become an increasingly important tool. For example, measurement of gene expression has been used in several different applications to monitor biological responses to various stimuli. Several different approaches are currently available to make quantitative determinations of nucleic acids, but after two decades the polymerase chain reaction (PCR) remains a core tool for molecular biology. PCR is now an essential element in clinical diagnostics and environmental detection, and has also been applied to nearly every approach in molecular biology including site directed mutagenesis, subtractive hybridization, library construction, and numerous other routine applications such as cloning and sequencing.

For example, recently developed PCR-based mutation detection assays offer highly reliable methods of identifying a single nucleotide variation in a given fragment. As a diagnostic tool, this offers the powerful advantage of allowing one to conveniently prescreen large numbers of unknown samples of which only implicated variants would need to be directly sequenced. Based on the ability to easily identify repeated polymorphisms of high copy number, DNA typing methods allow questions of simple genetic linkage, paternity, evolutionary taxonomy, and population genetics to be conveniently addressed through a simple assay.

The need for accurate, reliable, and efficient methods of individual identification, for example in diagnostics or forensics, has spurred rapid growth in the identification of polymorphic sequences that are now used in numerous DNA typing methods. The rapid diversification of these techniques also reflects the growing value of being able to type genomic DNA in a wide range of organisms and their subspecies. The discovery of small, abundant, highly polymorphic repetitive sequences is the critical basis of powerful genetic typing methods, and PCR has led to dramatic innovations in this field. The use of ever smaller polymorphic sequences combined with sensitive PCR-based sequence amplification techniques has significantly reduced the restrictions of sample quality, quantity, and sensitivity that were early obstacles to the rapid development and application of these methods.

The great advantage that PCR has provided over traditional cloning methods is the selective amplification of a specific sequence in a population of many. This has been the basis for quantitation of transcripts in cDNA populations; for the identification of microbial sequences in clinical or environmental samples; the detection of allelic variants; and the use of primers to introduce genetic modification. However, the remarkable ability of PCR to amplify specific DNA sequences has, along with its obvious benefits, some practical pitfalls that require careful attention. First among these is the ability of PCR to amplify DNA inadvertently introduced into the reaction. Precautions against contamination are especially important in forensic and clinical applications, but must be considered in every laboratory using the technique. Some principles used in sterile culture of microorganisms are applicable, but additional precautions, such as strict segregation of sample preparation, reaction assembly, thermocycler, and analysis work areas, and the use of positive displacement pipettes or aerosol preventive tips, may also be necessary. The use of ultraviolet light and chemical decontamination procedures as well as of enzymatic methods to prevent the amplification of “carryover” templates should be employed in some situations.

Even without contamination, amplification of DNA templates in clinical and research settings are often from a mixture of DNAs, with the target DNA making up a small minority. The specificity of the amplification is typically determined by construction of specific primers targeted to a particular template. Therefore, templates that differ in sequence but have identical primer binding sites are not easily distinguished during the amplification process. An inherent limitation of current amplification reactions is the inability to control amplification success among such templates. All templates that match the primers can amplify, but templates that are shorter, more numerous, or amplify more easily can quickly dominate a reaction.

A great deal of effort is expended attempting to circumvent specific aspects of this general problem. Elaborate procedures to either avoid or remove laboratory contaminants are one such manifestation. Optimization of the PCR reaction to reduce or eliminate unwanted products, such as primer-dimers and additional bands, are also attempts to reduce the impact of this lack of control. The design and use of specific primers may be the most common approach to circumvent this problem by including a second set of primers, which can provide sufficient discrimination. However, specific primers require extensive a priori sequence knowledge, making them impractical for acquiring unknown sequences, and they change the resulting product such that direct comparisons among primer sets are problematic. Thus they are not suitable for many applications.

An alternative approach is to modify the PCR conditions to add an additional level of amplification control while retaining the simplicity and efficiency of the standard PCR methodology. Preferably, a method of this type could be utilized in a variety of PCR reactions. These needs are addressed by the present invention.

Publications

Yuen et al. (2001) Nucleic Acids Res 29: e31 discusses inhibition of PCR amplification by degenerate primers. Chen and Ruffner (1998) Nucleic Acids Res26(4):1126-7 describe an in vitro procedure, ligation-during-amplification (LDA), for selective amplification of closed circular DNA using sequence-specific primers. Michael et al. (1994) Biotechniques 16(3):410-2 describe mutagenesis by incorporation of a phosphorylated oligo during PCR amplification.

### SUMMARY OF THE INVENTION

Methods and compositions are provided for template specific inhibition during PCR (TSI-PCR), which allows specific inhibition of particular templates without disrupting the amplification of other templates with similar amplification primer binding sites. TSI-PCR is achieved during polymerase chain reaction by the inclusion of modified oligonucleotides, herein termed stop oligos. The stop oligos comprise a 3′ modification that prevents extension by DNA polymerases, and a 5′ phosphate. Stop oligos hybridize to a region of the amplicon, i.e. are complementary to a region of polynucleotide that lies between the two PCR amplification primers.

In the methods of the invention, a PCR reaction mixture is utilized, comprising a complex pool of target polynucleotides; one or more stop oligos; and a DNA ligase, usually a temperature stable template dependent DNA ligase. The stop oligo sequence will hybridize to amplicons having complementary sequences. During PCR amplification, when the extending DNA strand reaches the point where the stop oligo is bound, the newly formed strand is ligated to the stop oligo. The 3′ modification to the stop oligo blocks further extension, truncating that strand. Because the truncated products are not full length, and cannot be extended, they do not serve as additional templates during subsequent rounds of amplification. The amplicons complementary to a stop oligo are therefore specifically inhibited over multiple amplification cycles. PCR will continue to amplify non-complementary amplicons.

The method of the invention find use in a variety of techniques where it is desirable to amplify templates from a complex target population, particularly where multiple target polynucleotides hybridize to common amplification primers, e.g. in the detection of allelic variants; the identification of bacterial subspecies; directed mutagenesis; and the like. The methods of the invention provide the advantage that a priori sequence knowledge is required only for the target population to be inhibited, allowing recovery of previously unknown minority templates with existing primers.

In one embodiment of the invention, a reaction mixture is provided for template specific inhibition during PCR, where the reaction mixture comprises at least one stop oligo; PCR primers; thermostable DNA polymerase; thermostable DNA ligase; and deoxynucleoside triphosphates (dNTPS) for all four deoxynucleotides. Kits for the practice of the methods of the invention are also provided.

### BRIEF DESCRIPTION OF THE DRAWINGS

FIG. 1. Schematic of TSI-PCR reaction. Heat denaturation of template DNA results in single stranded templates. Amplification primers and stop oligos anneal to single stranded templates in a sequence specific manner, and primers are extended by DNA polymerase until the extending strand reaches the stop oligo. Then the extending strand is ligated to the stop oligo by a thermotolerant ligase effectively blocking further extension. The result is linear amplification of the truncated product and no amplification of full length template when the stop oligo matches the template. Templates that do not match stop oligos amplify geometrically as in a typical PCR reaction.

FIG. 2. Effect of different amplification conditions for single template amplifications. Lanes 1-3 used 0.5 pg B. glandula plasmid as template. Lanes 4-6 used 0.5 pg E. mathaei plasmid as template. Amplifications were performed with three regimes; Lanes 1 and 4 represent TSI-PCR conditions with stop oligos designed to inhibit the B. glandula template. Stop oligos were omitted from the reaction mix for samples shown in lanes 2 and 5. Lanes 3 and 6 show products of reactions that contained all the TSI-PCR components except ligase.

FIG. 3. Demonstration of uninhibited (no ligase) and TSI-PCR reactions using mixed templates composed of a constant 0.25 pg of E. mathaei template per reaction and a gradient of undesired, B. glandula, template from 0.25 pg to 2.5 ng which represents 1:1 to 1:10,000 ratios of the two templates, and results of Apo I digestion of the resulting amplification products. M denotes 1 kb DNA ladder (BRL) with approximate band sizes indicated. a) Amplification products from uninhibited (no ligase) reactions. Lane 1, 1:1 starting ratio of the two templates; Lane 2, 1:10 ratio; Lane 3, 1:100 ratio; Lane 4, 1:1000 ratio; Lane 5, 1:10,000 ratio. b) Apo I digests of the products from lanes 1-5. c) Amplification products from TSI-PCR reactions. Lane 11, 1:1 ratio; Lane 12, 1:10 ratio; Lane 13, 1:100 ratio; Lane 14, 1:1000 ratio; Lane 15, 1:10,000 ratio. d) Apo I digests of the products from lanes 11-15.

FIG. 4. Comparison of percentage of clones derived from desired E. mathaei templates versus undesired B. glandula templates at different starting concentrations of undesired template. In each experiment 0.25 pg of E. mathaei template was added. Two sets of stop oligos were used, Stop 1F+3R (boxes) or Lig 1+2 (triangles) either utilizing TSI-PCR reaction conditions (solid symbols) or no ligase control reactions similar to standard PCR (empty symbols).

### DEFINITIONS

The terms “nucleoside” and “nucleotide” are intended to include those moieties that contain not only the known purine and pyrimidine bases, but also other heterocyclic bases that have been modified. Such modifications include methylated purines or pyrimidines, acylated purines or pyrimidines, alkylated riboses or other heterocycles. In addition, the terms “nucleoside” and “nucleotide” include those moieties that contain not only conventional ribose and deoxyribose sugars, but other sugars as well. Modified nucleosides or nucleotides also include modifications on the sugar moiety, e.g., wherein one or more of the hydroxyl groups are replaced with halogen atoms or aliphatic groups, or are functionalized as ethers, amines, or the like.

A “nucleic acid” is a deoxyribonucleotide or ribonucleotide polymer in either single or double-stranded form, including known analogs of natural nucleotides unless otherwise indicated.

A “polynucleotide” refers to a single or double-stranded polymer of deoxyribonucleotide or ribonucleotide bases. An “oligonucleotide” refers to a single or dbuble-stranded polymer of deoxyribonucleotide or ribonucleotide bases, usually a single stranded polymer, which is less than about 100 nt in length, usually less than about 50 nt. in length, and includes probes and primers, as described below. Analogs of natural oligonucleotides may also be used, for example incorporating non-natural bases, linkage, sugars, and the like.

“Terminator nucleotide” refers to a nucleotide that prevents elongation of a polynucleotide chain. In one embodiment, a terminator nucleotide contains a chemical modification at the 3′ end that prevents normal polymerization of the nucleotide into a polymer, e.g. polymerization by DNA polymerase. Such terminator nucleotides may retain the ability to form base pairs, and may be recognized by enzymes that act on polynucleotides. In another embodiment, polymerization is inhibited by inclusion of a deliberate mismatch or mismatches at the terminus of an otherwise complementary polynucleotide.

Such terminator modifications are known in the art, and include, without limitation: 2′,3′ dideoxythymidine; 2′,3′ dideoxycytidine; 2′,3′ dideoxyuridine; 2′,3′ dideoxyguanosine; 2′,3′ dideoxyadenosine. Any of the bases may be modified by addition of an alkyl spacer at the 3′ end, which inactivates the 3′ OH towards enzymatic processing. One of skill in the art will recognize that such spacers may be variable in the length of the carbon chain, e.g. 1, 2, 3, 4, 5 carbons, etc. Inverted bases, such as inverted dT, when incorporated at the 3′-end of an oligo lead to a 3′-3′ linkage which inhibits both degradation by 3′ exonucleases and extension by DNA polymerases. 3′-O-methyl-dNTPs are described by Metzker et al. (1994) Nucleic Acids Res. 22(20):4259-4267. A large number of other modified or capped nucleotides have been described in the art, and may be used in the methods of the invention.

A “probe” is a nucleic acid capable of binding to a target nucleic acid of complementary sequence through base pairing, thus forming a duplex structure. The probe binds or hybridizes to a “probe binding site.” A probe may include natural (i.e. A, G, C, or T) or modified bases (7-deazaguanosine, inosine, etc.). A probe can be an oligonucleotide that is a single-stranded DNA. Oligonucleotide probes can be synthesized or produced from naturally occurring polynucleotides. Some probes may have leading and/or trailing sequences of noncomplementarity flanking a region of complementarity.

A “perfectly matched probe” has a sequence perfectly complementary to a particular target sequence. The probe is typically perfectly complementary to a portion (subsequence) of a target sequence. The term “mismatched probe” refer to probes whose sequence is not perfectly complementary to a particular target sequence, but which retains sufficient complementary to bind under less stringent conditions.

A “primer” is a single-stranded oligonucleotide capable of acting as a point of initiation of template-directed DNA synthesis under appropriate conditions (i.e., in the presence of four different nucleoside triphosphates and an agent for polymerization, such as, DNA or RNA polymerase or reverse transcriptase) in an appropriate buffer and at a suitable temperature. Primers of the invention include amplification primers, and stop oligos. The appropriate length of a primer depends on the intended use of the primer but is usually sufficient to provide for hybridization under the desired conditions, and is usually at least about 12 nucleotides in length, at least about 15 nucleotides in length, at least about 18 nucleotides in length, at least about 20 nucleotides in length, and usually not more than about 40 nucleotides in length, or not more than about 30 nucleotides in length. Short primer molecules generally require cooler temperatures to form sufficiently stable hybrid complexes with the template. A primer need not reflect the exact sequence of the template but must be sufficiently complementary to hybridize with a template. The term “primer site” refers to the area of the target DNA to which a primer hybridizes. The term “primer pair” means a set of primers including a 5′ “upstream primer” that hybridizes with the 5′ end of the DNA sequence to be amplified and a 3′ “downstream primer” that hybridizes with the complement of the 3′ end of the sequence to be amplified.

The primers and oligos described above and throughout this specification may be prepared using any suitable method, such as, for example, the known phosphotriester and phosphite triester methods, or automated embodiments thereof. In one such automated embodiment, dialkyl phosphoramidites are used as starting materials and may be synthesized as described by Beaucage et al (1981), Tetrahedron Letters 22, 1859. One method for synthesizing oligonucleotides on a modified solid support is described in U.S. Pat. No. 4,458,066.

The term “substantially complementary” means that a primer or probe need not be exactly complementary to its target sequence; instead, the primer or probe need be only sufficiently complementary to selectively hybridize to its respective strand at the desired annealing site. A non-complementary base or multiple bases can be included within the primer or probe, so long as the primer or probe retains sufficient complementarity with its polynucleotide binding site to form a stable duplex therewith.

The term “stringent assay conditions” as used herein refers to conditions that are compatible to produce binding pairs of nucleic acids, e.g., surface bound and solution phase nucleic acids, of sufficient complementarity to provide for the desired level of specificity in the assay while being less compatible to the formation of binding pairs between binding members of insufficient complementarity to provide for the desired specificity. Stringent assay conditions are the summation or combination (totality) of both hybridization and wash conditions. Low stringency hybridization conditions in the context of nucleic acid hybridization (e.g., as in array, Southern or Northern hybridizations) are sequence dependent, and are different under different experimental parameters. The specific temperature and salt concentrations for the reaction may be tailored to capture the sequences of interest. An example of low stringency conditions includes hybridization in a buffer comprising 5×SSC and 1% SDS at from about 20 to about 42° C., with a wash of 0.2×SSC and 0.1% SDS at from about 20 to about 42° C. Exemplary high stringency hybridization conditions may include hybridization in a buffer of 40% formamide, 1 M NaCl, and 1% SDS at 37° C., and a wash in 1×SSC at 45° C. Stringent hybridization conditions also include hybridization at 60° C. or higher and 3×SSC (450 mM sodium chloride/45 mM sodium citrate) or incubation at 42° C. in a solution containing 30% formamide, 1M NaCl, 0.5% sodium sarcosine, 50 mM MES, pH 6.5. Those of ordinary skill will readily recognize that alternative but comparable hybridization and wash conditions can be utilized to provide conditions of similar stringency.

The term “amplify” in reference to a polynucleotide means to use any method to produce multiple copies of a polynucleotide segment, called the “amplicon” or “amplification product” , by replicating a sequence element from the polynucleotide or by deriving a second polynucleotide from the first polynucleotide and replicating a sequence element from the second polynucleotide. The copies of the amplicon may exist as separate polynucleotides or one polynucleotide may comprise several copies of the amplicon. The precise usage of amplify is clear from the context to one skilled in the art.

A preferred amplification method utilizes PCR (see Saiki et al. (1988) Science 239:487-4391). The method utilizes a pair of primers that flank the desired target sequence, and may be specific or degenerate. In conventional PCR the primers are mixed with a solution containing the target DNA (the template), a thermostable DNA polymerase and deoxynucleoside triphosphates (dNTPS) for all four deoxynucleotides. The mix is then heated to a temperature sufficient to separate the two complementary strands of DNA. The mix is next cooled to a temperature sufficient to allow the primers to specifically anneal to sequences flanking the gene or sequence of interest. The temperature of the reaction mixture is then optionally reset to the optimum for the thermostable DNA polymerase to allow DNA synthesis (extension) to proceed. The temperature regimen is then repeated to constitute each amplification cycle. Thus, PCR consists of multiple cycles of DNA melting, annealing and extension.

The PCR methods used in the methods of the present invention are carried out using standard methods (see, e.g., McPherson et al., PCR (Basics: From Background to Bench) (2000) Springer Verlag; Dieffenbach and Dveksler (eds) PCR Primer: A Laboratory Manual (1995) Cold Spring Harbor Laboratory Press; Erlich, PCR Technology, Stockton Press, New York, 1989; Innis et al., PCR Protocols: A Guide to Methods and Applications, Academic Press, Harcourt Brace Javanovich, New York, 1990; Barnes, W. M. (1994) Proc Natl Acad Sci U S A, 91, 2216-2220). The primers and oligonucleotides used in the methods of the present invention are preferably DNA and analogs thereof, e.g. phosphorothioates; phosphorodithioates, where both of the non-bridging oxygens are substituted with sulfur; phosphoroamidites; alkyl phosphotriesters and boranophosphates. Achiral phosphate derivatives include 3′-O′-5′-S-phosphorothioate, 3′-S-5′-O-phosphorothioate, 3′-CH₂-5′-O-phosphonate and 3′-NH-5′-O-phosphoroamidate. Such nucleic acids can be synthesized using standard techniques

### DETAILED DESCRIPTION OF THE EMBODIMENTS

Before the subject invention is described further, it is to be understood that the invention is not limited to the particular embodiments of the invention described below, as variations of the particular embodiments may be made and still fall within the scope of the appended claims. It is also to be understood that the terminology employed is for the purpose of describing particular embodiments, and is not intended to be limiting. Instead, the scope of the present invention will be established by the appended claims.

In this specification and the appended claims, the singular forms “a,” “an” and “the” include plural reference unless the context clearly dictates otherwise. Unless defined otherwise, all technical and scientific terms used herein have the same meaning as commonly understood to one of ordinary skill in the art to which this invention belongs.

Where a range of values is provided, it is understood that each intervening value, to the tenth of the unit of the lower limit unless the context clearly dictates otherwise, between the upper and lower limit of that range, and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood to one of ordinary skill in the art to which this invention belongs. Although any methods, devices and materials similar or equivalent to those described herein can be used in the practice or testing of the invention, the preferred methods, devices and materials are now described.

All publications mentioned herein are incorporated herein by reference for the purpose of describing and disclosing the invention components that are described in the publications that might be used in connection with the presently described invention.

### TSI-PCR

Methods and compositions are provided for amplification of targeted polynucleotide sequences in a PCR reaction from a complex population of templates, where two or more templates are amplified by the same primers. The pool of amplification primers may have a single specificity, or may contain degeneracies allowing hybridization to multiple targets to provide for a greater range of targets. The desired templates for amplification, and templates targeted for inhibition, will each have a site for hybridization of the amplification primers, although the sites need not be identical. Where the binding sites are non-identical, the amplification primers will contain sufficient degeneracy to bind to any desired template site and any inhibition-targeted template site.

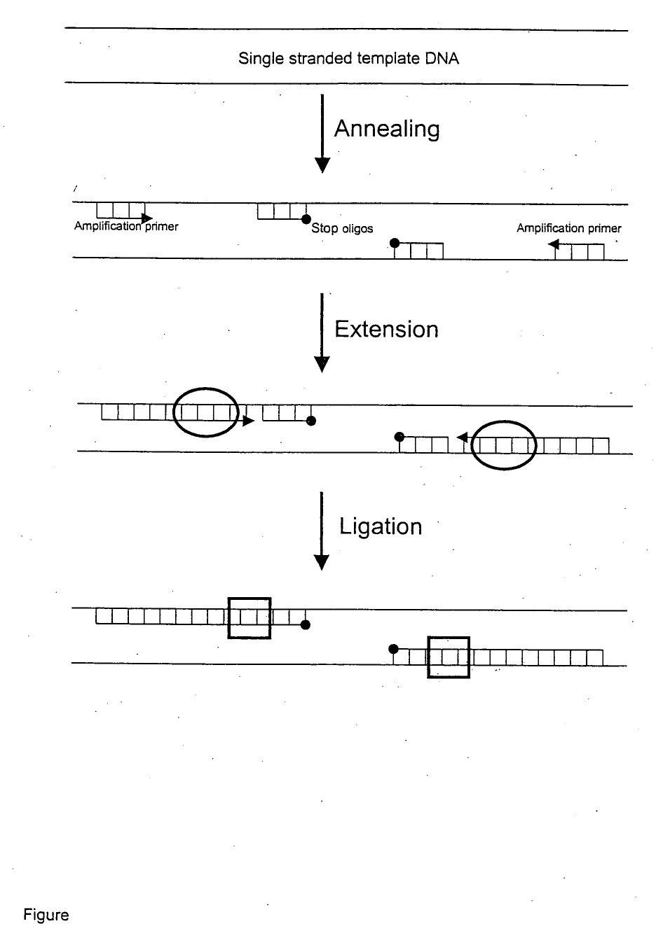

In addition to the reagents present in a conventional reaction mix, the TSI-PCR reaction mix also comprises a stop oligo, which comprises a 3′ terminator nucleotide and a phosphorylated 5′ nucleotide. The stop oligo is complementary to a sequence of the template targeted for inhibition, and is not complementary to a sequence of the desired template. The complementary sequence to the stop oligo may be any sequence that lies between the two amplification primers. It may hybridize to either strand of the amplicon, as shown in FIG. 1. The reaction may comprise a single stop oligo, or a cocktail of stop oligos, when it is desirable to inhibit a plurality of templates, or if redundancy for a single template is desired.

The reaction mix also comprises a thermostable DNA ligase. During PCR amplification, when the extending DNA strand reaches the point where the stop oligo is bound, the newly formed strand is ligated to the stop oligo. The 3′ modification to the stop oligo blocks further extension, truncating that strand. Because the truncated products are not full length, and cannot be extended, they do not serve as additional templates during subsequent rounds of amplification. The amplicons complementary to the stop oligo are therefore specifically inhibited over multiple amplification cycles. PCR will continue to amplify non-complementary amplicons.

### Reactions

The methods of the invention are applicable to the amplification of any complex template population where more than one template can be amplified in the reaction. In some embodiments, alleles or mutations or other genetic variability in a population is assessed. For example, a predominant sequence in a population may be targeted for inhibition, in order to allow amplification of minor species.

In other embodiments, the methods are useful in analysis of polynucleotides obtained from complex populations, such as microbial communities in clinical samples; environmental samples, e.g. ground water, sea water, mining waste, etc.; biological samples, e.g. lysates prepared from crops, tissue samples, etc.; manufacturing samples, e.g. time course during preparation of pharmaceuticals; and the like.

From the source of interest, a nucleic acid sample population is provided. If double stranded, the nucleic acid is first denatured to form single stranded nucleic acid using any of a variety of denaturation techniques which are known in the art, including, for example, physical, chemical, enzymatic or thermal means. Typically, strand separation is achieved using heat denaturation at temperatures ranging from 80° C. to about 105° C. for time periods ranging from about 1 to 10 minutes. For cases in which the nucleic acid is RNA, the sample may first be reverse transcribed to form cDNA, which is then denatured.

The amplification is performed in a TSI-PCR reaction mix. The mix comprises at least one stop oligo, as described above. The concentration of stop oligo may be empirically determined, depending on the concentration of template DNA, but conveniently is at least about 0.1 μM to about 1 mM, usually from about 1 μM to about 250 μM; more usually from about 25 μM to about 75 μM.

The stop oligo comprises a termination nucleotide at the 3′ end, and is phosphorylated at the 5′ end. Phosphorylation is accomplished by any convenient method as known in the art, for example reaction with DNA kinase. The stop oligo may be a single sequence, or a cocktail of sequences, e.g. including degenerate positions. The sequence is complementary to a region of the amplicon that is targeted for inhibition. Selection of sequences for primer design is known in the art, and is conveniently performed with any of a variety of software packages designed for that purpose. The specific location of the stop oligo target sequence is not critical to the sequence, so long as it lies between the two amplification primers.

The reaction mix will further comprise a thermostable DNA ligase, at a concentration effective to ligate the stop oligos to nascent amplicon sequences. Such ligases are known in the art and commercially available, e.g. Taq DNA ligase (new England Biolabs); Tsc Ligase (Roche Biosciences). Preferably the DNA ligase is present in the initial reaction mixture.

The denatured template strands are incubated with amplification primers, and stop oligos, under hybridization conditions, i.e., conditions in which the primers, and stop oligos anneal to their respective complementary portions of the single stranded nucleic acid. Primers are selected so that the primer binding sites to which they anneal are located so as to result in the formation of an extension product which, once separated from its template strand, can itself serve as a template for extension by the other primer.

Because the denatured nucleic acid strands are typically considerably longer than the primers and stop oligos, there is an increased probability that a denatured strand makes contact and reanneals with its complementary strand before the primer or probe has a chance to hybridize to their complementary sequences. To avoid this problem, a high molar excess of primer and stop oligos are used to increase the likelihood that they anneal to their respective template strand before the denatured strands reanneal.

As is typical of amplification methods, two primers (a primer pair) are used to amplify the nucleic acid sequence of interest. The primers used in the amplification are selected so as to be capable of hybridizing to sequences at flanking regions of the target being amplified. The primers are chosen to have at least substantial complementarity with the different strands of the nucleic acid being amplified. The primer must have sufficient length so that it is capable of priming the synthesis of extension products in the presence of an agent for polymerization. The length and composition of the primer depends on many parameters, including, for example, the temperature at which the annealing reaction is conducted, proximity of the probe binding site to that of the primer, relative concentrations of the primer and probe and the particular nucleic acid composition of the probe. Typically the primer comprises from around about 15 to not more than about 30 nucleotides. However, the length of the primer may be more or less depending on the complexity of the primer binding site and the factors listed above. Each primer may comprise a single sequence, or a cocktail of sequences.

The amplification reaction is performed under conventional conditions, and may utilize a 3 temperature/3 step cycle; or a 2 temperature/2 step cycle. The reaction may be run for any number of cycles, usually at least about 5, more usually at least about 10, and may be 20 cycles, 30 cycles, or more.

During the amplification process, those amplicons that are complementary to the stop oligo are inhibited from further extension, thereby allowing preferential amplification of the non-complementary amplicons. The final amplification product will comprise a majority of sequences that are non-complementary to the stop oligo.

The amplification product may be used in various methods, e.g. sequencing, cloning, hybridization to arrays, blots, and the like, using all techniques known to those of skill in the art. For hybridization purposes, it may be desirable to introduce detectable labels, e.g. fluorochromes; radioactive label; epitope tags, and the like. For cloning purposes, linkers may be ligated to the product; and other manipulation steps as used in the art.

### Kits

Also provided are kits for use in the subject invention, where such kits may comprise containers, each with one or more of the various reagents/compositions utilized in the methods, where such reagents/compositions typically at least include stop oligos or termination nucleotides useful in the synthesis of stop oligos, and reagents employed in nucleic acid amplification, e.g., primers, buffers, the appropriate nucleotide triphosphates (e.g. dATP, dCTP, dGTP, dTTP), DNA polymerase, labeling reagents, e.g., labeled nucleotides, and the like. Alternatively, kits may include buffers, enzymes, including enzyme blends, and instructions, where the customer provides specific primers.

Finally, the kits may further include instructions for using the kit components in the subject methods. The instructions may be printed on a substrate, such as paper or plastic, etc. As such, the instructions may be present in the kits as a package insert, in the labeling of the container of the kit or components thereof (i.e., associated with the packaging or sub- packaging) etc. In other embodiments, the instructions are present as an electronic storage data file present on a suitable computer readable storage medium, e.g., CD-ROM, diskette, etc.

The following examples are put forth so as to provide those of ordinary skill in the art with a complete disclosure and description of how to make and use the subject invention, and are not intended to limit the scope of what is regarded as the invention. Efforts have been made to ensure accuracy with respect to the numbers used (e.g. amounts, temperature, concentrations, etc.) but some experimental errors and deviations should be allowed for. Unless otherwise indicated, parts are parts by weight, molecular weight is average molecular weight, temperature is in degrees centigrade; and pressure is at or near atmospheric.

### Experimental

Normal PCR achieves amplification of a specific minor template only if its sequence is known and can be used to make specific oligonucleotide primers. The methods of the invention provide an alternative approach. TSI-PCR inhibits amplification of known elements of a mixture, allowing different, and potentially unknown, templates to be amplified and recovered.

Materials and Methods

To investigate the ability of the TSI-PCR reactions to specifically inhibit a particular template without disrupting the amplification of other templates utilizing the same primers, we utilized a simple two template amplification system. We utilized templates of known concentration and identical primer binding sites already available in our lab for convenience. We arbitrarily designated one of these templates, derived from the 18S ribosomal subunit gene of the barnacle Balanus glandula, as undesired and designed stop oligos against this template. The other 18S template, derived from the sea urchin Echinometra mathaei, was therefore the desired template. We estimated the inhibitory effect of TSI-PCR on these templates separately, and also by using mixed template starting material. We also used increasing amounts of undesired template in order to test the amount of inhibited template that can be effectively blocked from amplification. Additionally, sequence specificity of the stop oligos was determined by comparing the ability of TSI-PCR to distinguish templates differing by only a single nucleotide in the oligo binding region.

Primer and template design. Table 1 lists the sequences for the oligonucleotides used in the TSI-PCR reactions and construction of plasmid templates. Two sets of amplification primers for TSI-PCR were used; either the 18S rRNA gene primers 515F1 and 1209R6 or the vector primers.SP6 and T7. Two sets of stop oligos, B.glan stop 1F and B.glan stop 3R or Lig 1 and Lig 2, were also used to assure that the observed inhibition could be universally achieved. All of the stop oligos were designed to bind specifically to a portion of the 18S rRNA gene from the barnacle B. glandula, and included 3′ base modifications that inhibit extension by DNA polymerases (Table 1). Stop oligos Lig 1 and Lig 2 were synthesized with 5′ phosphorylation, B.glan stop 1F and B.glan stop 3R were 5′ phosphorylated with T4 polynucleotide kinase (New England Biolabs) before use.

Templates used in TSI-PCR were plasmid constructs of known concentration. Plasmids were constructed by TA cloning of PCR generated framents of the 18S rRNA gene from B. glandula or E. mathei into the Pgem-T vector (Promega). Plasmids were isolated by Wizard Prep (Promega) alkaline lysis minipreps from overnight cultures derived from single E. coli clones. The DNA concentration was quantified by spectrophotometer at 260 nm.

Four separate plasmid templates were constructed. The B. glandula and E. mathaei plasmids contain the inserts resulting from PCR amplification of B. glandula and E. mathaei genomic DNA respectively with the 18S rRNA primers 515F1 and 1209R6. These plasmids therefore have incorporated identical sequences corresponding to the primers 515F1 and 1209R6, but differ at numerous base positions between these primers corresponding to differences in their genomic templates. Amplifications from these templates were conducted using 515F1 and 1209R6 as amplification primers and either B.glan stop 1F and B.glan stop 3R as stop oligos or Lig 1 and Lig 2 as stop oligos.

The additional plasmids contain inserts derived by amplifying B. glandula genomic DNA. One product was amplified with primers Match 1F and Match 2R which are both perfect matches to the B. glandula template. Thus, the resulting plasmids contain perfect matches to the Lig 1 and Lig 2 stop oligos. The other insert was amplified with primers Mod 1F and Mod 2R, that have single base mismatches to the 5′ nucleotides of the Lig 1 and Lig 2 stop oligos respectively (see Table 1). The Mod 1F and Mod 3R primers also extend several bases upstream (5′) of the Lig 1 and Lig 2 primers such that the resulting plasmid is a total of 15 nucleotides larger than the plasmid derived from the Match 1F and 2R amplicon. The additional 7 upstream bases of the Mod 1F primer served not only to anchor the mutagenic primer to the B. glandula template, but also to retain a unique BamHI restriction site present in that template which facilitates subsequent screening. Amplifications from these templates were conducted using vector primers SP6 and T7 as amplification primers and either Lig 1 and Lig 2 as stop oligos, or Lig 1 as a single stop oligo.

Table 1. Sequence composition of oligonucleotides used. Asterisk denotes 5′ phosphorylated bases. Underlined bases denote locations of introduced single base mismatches to the Lig 1 and Lig 2 stop oligos.

TABLE 1

| | | |
|---|---|---|
| Name | Sequence | Length |

| | | | |
|---|---|---|---|
| M13R M13F SP6 | SEQ ID NO:1 | 5′-GGAAACAGCTATGACCATG-3′ | 19 bases |
| T7 | SEQ ID NO:2 | 5′-GTAATACGACTCACTATAG-3′ | 19 bases |
| 515F1 | SEQ ID NO:3 | 5′-GTGCCAGCAGCCGCGGTAA-3′ | 19 bases |
| 1209r6 | SEQ ID NO:4 | 5′-GGGCATCACAGACCTG-3′ | 16 bases |
| B. glan spec 1F | SEQ ID NO:5 | 5′-GGCGCTCACGCGTCACTGCT-3′ | 20 bases |
| B. glan spec 3R | SEQ ID NO:6 | 5′-GGCTGGGACGCCGATGAT-3′ | 18 bases |
| B. glan stop 1F | SEQ ID NO:7 | 5′-*GGCGCTCACGCGTCACTGCT(3′inverted dT)-3′ | 20 bases |
| B. glan stop 3R | SEQ ID NO:8 | 5′-*GGCTGGGACGCCGATGAT(3′inverted dT)-3′ | 18 bases |
| Lig 1 | SEQ ID NO:9 | 5′-*CTGGCGGGCCGTTCTTCG(3 C3 Spacer)-3′ | 18 bases |
| Lig 2 | SEQ ID NO:10 | 5′-*CCAACGGTCACAGGATTTCACC(3′Dideoxy C)-3′ | 22 bases |
| Match 1F | SEQ ID NO:11 | 5′-CTGGCGGGCCGTTCTTCG-3′ | 18 bases |
| Match 2R | SEQ ID NO:12 | 5′-CCAACGGTCACAGGATTTCACC-3′ | 22 bases |
| Mod 1F | SEQ ID NO:13 | 5′-GGATCCGATGGCGGGCCGTTC | 21 bases |
| Mod 2R | SEQ ID NO:14 | 5′-TTCGTCGTGCAACGGTCACAGG | 22 bases |

TSI-PCR reactions. The TSI-PCR was carried out in a 25 μl final volume using a 15 μl bottom mix containing 1× final concentration Stoffel buffer (Applied Biosystems), 20 nmol dNTPs, 1 U Amplitaq DNA polymerase stoffel fragment (Applied Biosystems), 25 nmol NAD, 0, 50, or 500 U of Taq DNA ligase (New England Biolabs), and from 0.5 pg to 2.5 ng plasmid template DNA. The additional 10 μl, containing 125 nmol MgCl₂, and all of the oligonucleotides was added during the initial 80° C. step to achieve a hot start. The oligonucleotides included 12.5 pmol each of two amplification primers and two stop oligos except as otherwise noted. The cycle parameters consisted of an initial one minute incubation at 80° C. followed by 95° C. for one minute, then 30 cycles of 95° C. for 30 seconds, and 60° C. for 12 minutes.

Single template amplifications were conducted using 0.5 pg of a single template. Mixed template reactions utilized 2 templates; one template matched the stop oligos, and the other had from one to several mismatches to the stop oligos. Mismatched templates were added at a constant amount of 0.25 pg per reaction, and the template with a perfect match to the stop oligos varied in amount by 4 orders of magnitude from 0.25 pg to 2.5 ng. This resulted in ratios of starting template from 1:1 to 1:10,000. Results of the TSI-PCR were visualized on an agarose gel. Amplicons resulting from B. glandula and E. mathaei derived templates were distinguished by restriction digestion with 2 U of Apo I at 50° C. for 3-6 hours. The Apo I cleaves E. mathaei but not B. glandula derived amplicons. Amplicons resulting from pMatch and pMod templates were distinguished by restriction digestion with 5 U of BamH I at 37° C. for 4 hours. Additionally, aliquots of some TSI-PCR reactions were ligated into the pGem-T vector (Promega) and transformed into competent E. coli by standard methods.

Screening. After transformation, colonies were screened for the presence of appropriate sized inserts by colony PCR using cycling parameters of 94° C. for two minutes followed by 30 cycles of 94° C. for 30 seconds, 55° C. for 30 seconds, and 72° C. for 60-90 seconds. The B. glandula and E. mathaei inserts were distinguished by using a multiplexed reaction that included the vector primers M13R and T7 as well as the B. glandula specific primers B.glan spec 1F and B.glan spec 3R that generate a smaller amplicon, but only on the B. glandula template. The B. glandula doublets were distinguished from the single E. mathaei band and other shorter length inserts by agarose gel electrophoresis.

Additionally, 16 Clones were sequenced to verify the identification based on the colony screens. The pMatch and pMod templates yielded similar sized products by colony PCR with SP6 and T7, but could be distinguished by the presence of a unique BamHI restriction site in amplicons generated from the pMod template. Duplicate 3 μl aliquots of successful colony screen reactions were incubated for 4 hours at 37 ° C. with or without the addition of 5 U BamHI in 1 μl 1× BamHI buffer (New England Biolabs). The pMod derived amplicons showed a mobility shift in the BamHI treatments relative to the untreated controls.

Results

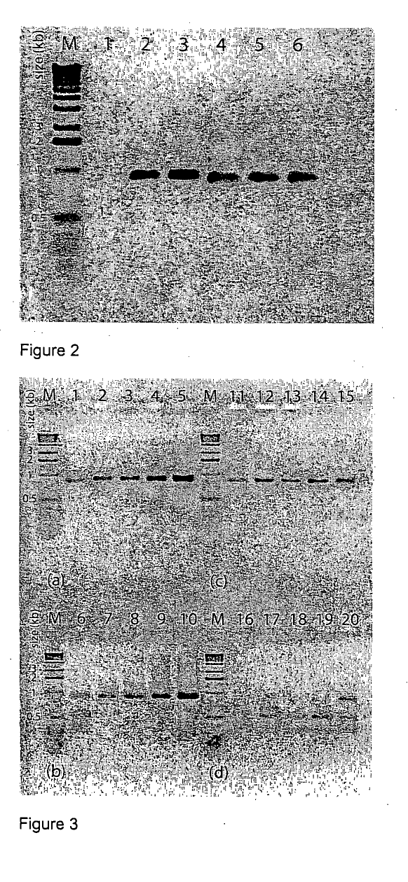

TSI-PCR in single template amplifications With equal concentrations of the B. glandula and E. mathaei templates in separate reactions, the TSI-PCR reaction appeared to completely inhibit amplification from the B. glandula template with no noticeable inhibition of amplification from the E. mathaei template (FIG. 2). Both templates amplified successfully when either the stop oligos or ligase were omitted (FIG. 2), demonstrating that the failure of the B. glandula TSI-PCR amplification is a property of the desired reaction conditions and not simply a failed amplification. Additionally, partial inhibition of amplification was observed for the B. glandula template when the stop oligo concentration was reduced by half, to 1.25 pmols, demonstrating that the stop oligos effect the inhibition in a concentration dependant manner. Both 50 U and 500 U of ligase were sufficient to achieve inhibition in this simplified system, although ligase concentration can alter inhibition success in some cases (see below).

TSI-PCR in mixed template amplifications The ability of the TSI-PCR to specifically inhibit the B. glandula template was investigated in a mixed template reaction using two separate sets of stop oligos both designed to be specific for the B. glandula template. The E. mathaei template was held constant at 0.25 pg while the B. glandula template varied by four orders of magnitude from 0.25 pg to 2.5 ng representing from 1:1 to 1:10,000 ratios of the respective templates. In the absence of ligase the intensity of the amplified bands reflected the concentration of total template DNA (FIG. 3). Digesting these products with Apo I, that cleaves only the E. mathaei product, had no visible effect except at the lowest B. glandula template concentration (FIG. 3). This is consistent with amplification of both templates roughly proportional to their starting concentrations. When TSI-PCR was activated by addition of ligase, the band intensities approximated those expected based on amplification of the E. mathaei template only (FIG. 3). Treatment of the TSI-PCR reactions with Apo I resulted in apparently complete digestion of the major band in all but the highest B. glandula template treatment, where only a faint band remained, suggesting amplification of the B. glandula template was specifically inhibited in these reactions (FIG. 3).

To quantitatively investigate the degree to which amplification of the B. glandula template was inhibited, the products resulting from TSI-PCR were cloned. Upon transformation, the resulting colonies were screened for whether they contained B. glandula or E. mathaei products. The ratios of clones mirrored the results seen with restriction digestion. Without ligase the ratio of products roughly followed the ratios of template amounts. At equal template concentrations, 20 of 42 colonies (48%) containing inserts of the appropriate size had B. glandula inserts when the first set of stop oligos were used, B. glan stop 1F and B. glan stop 3R, and 22 of 44 (50%) had B. glandula inserts when the second set of stop oligos were used, Lig 1 and Lig 2. With 250 pg of B. glandula template, approximately 1000 times more than the E. mathaei, 90 of 91 (99%) were B. glandula colonies with the first set of stop oligos, and all 85 were B. glandula with the second set. Likewise, 2.5 ng of B. glandula template resulted in all 57 and 71 colonies screened having B. glandula inserts.

Addition of ligase activated TSI-PCR conditions and essentially reversed those ratios (FIG. 3). Equal starting template concentrations resulted in all of the 24 and 89 colonies containing E. mathaei inserts when the first or second sets of stop oligos were used respectively. Even with the addition of 1000 fold excess (250 pg) of B. glandula template, 91 of 92 colonies (99%) contained a E. mathaei insert when the first set of stop oligos were used and all of the 33 colonies contained E. mathaei DNA with the second set of stop oligos. At the highest template amount, 2.5 ng, 44 of 51 colonies (86%) contained E. mathaei when the first set of stop oligos were used, and 38 of 42 colonies (90%) contained E. mathaei with the second set. Thus the vast majority of the colonies had E. mathaei inserts even when the starting concentration of B. glandula template was 10,000 times higher than the E. mathaei template (FIG. 3).

Additionally, the effect of ligase concentration on the efficacy of the TSI-PCR reaction was compared at the highest mixed template amount. Reducing the ligase concentration ten fold increased the number of B. glandula colonies slightly with 31 of 82 (38%) and 15 of 48 colonies (31%) having B. glandula inserts; however, this still represents a majority of E. mathaei clones despite a 10,000 fold excess of B. glandula template.

Specificity of stop oligos to 1 bp mismatches. To investigate the degree of specificity that can be achieved with TSI-PCR, the ability to differentially inhibit amplification of a template to which the stop oligos match perfectly, pMatch, was compared to a template that provides single base mismatches to the stop oligos, pMod. TSI-PCR amplifications using either a single stop oligo, Lig 1, or both Lig 1 and Lig 2 stop oligos resulted in substantial inhibition. Despite a 10,000 fold excess of the pMatch template, 19 of 48 colonies (40%) contained pMod products when Lig 1 was used as a single stop oligo, and 17 of 32 colonies (53%) contained pMod when both Lig 1 and Lig 2 were used to stop the reaction. With a 1000 fold excess of pMatch template 41 of 58 colonies (71 %) contained pMod products when Lig 1 was used as a stop oligo, and 40 of 42 colonies (95%) contained pMod when both Lig 1 and Lig 2 were used. The Lig 1 only reaction was also tested with a 100 fold excess of pMatch template, and all 48 clones screened contained the pMod product. The control reactions without the addition of ligase yielded nearly all pMatch products. A single clone, 1 of 35 (3%), had a pMod insert from the reaction using only the Lig 1 stop oligo and 1000 fold excess pMatch starting template concentration. The remainder of the clones from the control reactions contained pMatch products.

The TSI-PCR protocol expands upon the basic PCR framework to allow highly efficient user-directed inhibition of amplification of templates that have particular internal oligo binding sites. It adds a second level of specificity control to standard PCR by preventing amplification of templates that match a specific stop oligo. We have retained the amplification power of PCR for minor templates of unknown sequence by inhibiting the amplification of known, undesired, templates with the stop oligos. Desired templates utilizing the same amplification primers can be successfully amplified that would otherwise represent only minor components.

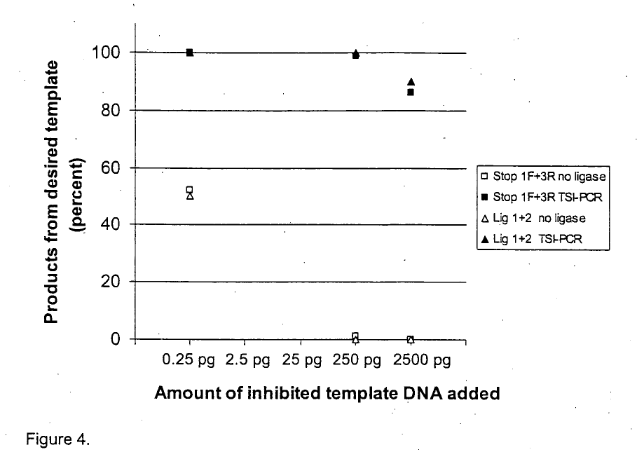

Using TSI-PCR, we show minority target templates can become a majority of the amplicons recovered despite as much as 10,000 fold excess of competing templates as starting DNA (FIG. 4). Without TSI-PCR conditions, the ratios of products recovered largely follow the ratios of input DNA suggesting the TSI-PCR is responsible for the shift. Thus TSI-PCR reactions have two determinants of specificity. Amplification primers define the set of potential amplicons that can be recovered and stop oligos inhibit amplification of a specific subset of those sequences.

The essence of TSI-PCR is simultaneous, competitive, extension by the DNA polymerase and ligation of partially extended products to the stop oligo (FIG. 1). We have demonstrated strong inhibition with two different sets of stop oligos and with different amplification primers. This suggests the inhibition is a robust and general property of these reactions rather than a specific phenomenon with a particular set of oligos.

This consistency of inhibition differs from previous approaches to selectively inhibit particular templates with oligonucleotides. Seyama et al. showed inhibition using Taq DNA polymerase and modified oligos, but this approach failed to yield inhibition in our hands. Yu et al. similarly failed to see inhibition using Taq, but was successful using the Stoffel fragment of DNA polymerase which lacks 5′-3′ exonuclease activity; however, this approximates the conditions of our control (no ligase) reactions for which we did not see detectable inhibition (FIGS. 2-4).

The peptide nucleic acid PCR clamping approach has found successful application, but the cost of PNA's and reduced inhibitory effect with PNA located apart from the amplification primers represent limitations on its widespread use. The reduced success of PCR clamping directed at internal sites is likely due to the truncated products that are formed. In subsequent rounds these truncated products may act as megaprimers that could competitively inhibit the binding of the peptide nucleic acids and can be extended to form full length products. In addition to leading to reduced inhibition of template, the megaprimers could also lead to other undesired outcomes such as in vitro recombination.

Without substantial optimization, TSI-PCR can achieve results comparable or better than those of PCR clamping with several additional benefits. The modified oligos utilized by TSI-PCR are more readily available and substantially lower in cost than peptide nucleic acids. Stop oligos can be directed at any portion of the internal sequence allowing much greater flexibility in distinguishing among templates. Because the truncated products produced in the TSI-PCR cycle have 3′ termini that prohibit further extension they cannot act as megaprimers in subsequent cycles. Thus TSI-PCR should be considerably less prone to PCR artifacts. Peptide nucleic acids typically provide greater single base pair specificity than DNA oligos, but TSI-PCR single base specificity takes advantage of the exquisite single base descrimination of Taq DNA ligase.

The specificity of NAD+ dependant ligases, such as Taq DNA ligase, is dependant upon the precise alignment of the 5′ phosphate and the adjacent 3′ hydroxyl group rather than overall oligo stability. Even single base pair mismatches up to nine bases away from the 5′ end can inhibit ligation. This is consistent with our finding that single base differences could be readily distinguished using TSI-PCR. However, the desired location of the mismatched bases for TSI-PCR differs from standard PCR primer design in that discriminating bases should be located near the 5′ end of the stop oligo rather than the 3′ end. Given the dynamics of primer-template binding, it seems logical that the stop oligos should be designed with a higher Tm than the amplification primers to insure a majority of the target templates have a stop oligo bound.

Other than the presumed benefit of a higher Tm and location of discriminating bases, the design of stop oligos is similar to the design of amplification primers. It is therefore likely that TSI-PCR will have the same flexibility to be adapted to nearly any set of sequences that specific primers have provided with standard PCR. We also expect a similar potential for multiplexing in order to remove several sequences simultaneously, and are currently exploring this possibility. Both the ligase and the stop oligos showed concentration dependant effects with decreases from our standard conditions, but we have not yet tested increasing the concentrations of stop oligos higher than the amplification primers, nor did we combine multiple sets of stop oligos directed at the same target simultaneously, although both could logically be expected to increase the degree of inhibition further.

For a majority of reactions we employed two stop oligos with one designed for each strand. To determine the ability of TSI-PCR to distinguish single base pair differences, we employed either one or two stop oligos under similar conditions. Overall the degree of inhibition of the undesired template was slightly lower for templates differing by a single base pair in the stop oligo recognition site than for templates differing by several bases. Reactions containing only one stop oligo showed a further reduction in ability to inhibit the undesired template against 1000 fold or higher excess of undesired template, but still resulted in a majority of the amplicons derived from the minority template at 1000 fold excess and 40% derived from the minority template despite a 10,000 fold excess of undesired template. Thus even a single stop oligo can provide substantial inhibition with single base pair discrimination.

One important feature of this approach is that it requires specific a priori information only for the inhibited template, not for the newly recovered sequences. Achieving additional specificity with the standard PCR approach requires designing more specific primers, which is difficult when the sequence of the target template is unknown. The TSI-PCR approach is different. It inhibits amplification of known elements of a mixture, allowing different, potentially unknown, minority templates to be amplified and recovered. The unique ability of TSI-PCR to provide robust sequence specific inhibition will have many uses in a variety of fields including DNA detection and amplification, clinical evaluation of mixed infections, genehunting, mutagenesis, cloning, and forensics. The TSI-PCR will likely have other applications where multiple templates are a problem. Unlike many contamination remedies, the TSI-PCR methodology allows for inhibition of undesired products whether the template is artificially introduced to the sample such as a laboratory contaminant, or is an intrinsic, but not desired, component of the sample as with mixed samples, presence of pseudogenes or gene families, or even multiple bands.

It is to be understood that this invention is not limited to the particular methodology, protocols, cell lines, animal species or genera, constructs, and reagents described, as such may, of course, vary. It is also to be understood that the terminology used herein is for the purpose of describing particular embodiments only, and is not intended to limit the scope of the present invention, which will be limited only by the appended claims.

Unless defined otherwise, all technical and scientific terms used herein have the same meaning as commonly understood to one of ordinary skill in the art to which this invention belongs. Although any methods, devices and materials similar or equivalent to those described herein can be used in the practice or testing of the invention, the preferred methods, devices and materials are now described.

All publications mentioned herein are incorporated herein by reference for the purpose of describing and disclosing, for example, the cell lines, constructs, and methodologies that are described in the publications, which might be used in connection with the presently described invention. The publications discussed above and throughout the text are provided solely for their disclosure prior to the filing date of the present application. Nothing herein is to be construed as an admission that the inventors are not entitled to antedate such disclosure by virtue of prior invention.

What is claimed is:

1. A method for template specific inhibition during polymerase chain reaction amplification, the method comprising:
amplifying a population of template polynucleotides in a reaction mix comprising:
amplification primers that flank a desired amplicon sequence present in at least a portion of said templates; at least one stop oligo complementary to a template targeted for inhibition; thermostable DNA polymerase; and thermostable DNA ligase;
wherein ligation of said stop oligo to polymerization products complementary to said template targeted for inhibition block amplification, while permitting amplification of non-targeted amplicons.

2. The method according to claim 1, wherein said at least one stop oligo comprises a 3′ modification resistant to polymerase extension.

3. The method according to claim 2, wherein said 3′ modification is selected from a dideoxy modification; a 3′ alkyl spacer; or an inverted base.

4. The method according to claim 2, wherein said stop oligo comprises a single nucleotide species.

5. The method according to claim 2, wherein said stop oligo comprises a plurality of nucleotide species.

6. A reaction mixture for template specific inhibition during polymerase chain reaction amplification, comprising:
amplification primers that flank a desired amplicon sequence present in at least a portion of said templates; at least one stop oligo complementary to a template targeted for inhibition; thermostable DNA polymerase; and thermostable DNA ligase.

7. The reaction mixture according to claim 6, wherein said at least one stop oligo comprises a 3′ modification resistant to polymerase extension.

8. The reaction mixture according to claim 7, wherein said 3′ modification is selected from a dideoxy modification; a 3′ alkyl spacer; or an inverted base.

9. The reaction mixture according to claim 7, wherein said stop oligo comprises a single nucleotide species.

10. The reaction mixture according to claim 7, wherein said stop oligo comprises a plurality of nucleotide species.

11. A kit for template specific inhibition during polymerase chain reaction amplification, the kit comprising:
termination nucleotides, and reagents employed in nucleic acid amplification.
